(19) United States
(12) Patent Application Publication
(10) Pub. No.: US 2005/0281791 A1
(43) Pub. Date: Dec. 22, 2005

(54) Method for inducing cardiac myocytes in embryonic stem (ES) cells by induction with precardiac endoderm and mesoderm

(75) Inventors: John William Lough, Elm Grove, WI (US); Diane Elizabeth Rudy-Reil, Plymouth, WI (US)

Correspondence Address: MARJORY S. STEWART LAW OFFICE, 611 NORTH BROADWAY, SUITE 510, MILWAUKEE, WI, 53202, US

(73) Assignee: Specialized Stem Cells, LLC

(21) Appl. No.: 11/159,567

(22) Filed: Jun. 22, 2005

Related U.S. Application Data
(60) Provisional application No. 60/581,946, filed on Jun. 22, 2004.

(51) Int. Cl. A61K 48/00; C12N 5/08

(52) U.S. Cl. 424/93.21; 435/366

(57) ABSTRACT

A method to induce ES cells to the cardiac phenotype is disclosed whereby avian precardiac endoderm used as feeder/inducer cells induce high percentage conversion of mouse embryonic stem (mES) cells into cardiac myocytes. Upon induction, the majority (˜65%) of co-cultured ES cell-derived embryoid bodies (EBs) become enriched in cardiac myocytes and exhibit rhythmic contractions. When precardiac mesoderm is included with the precardiac endoderm, ˜100% of EBs become rhythmically contractile. The inductive effect of the precardiac endoderm/mesoderm is mimicked by medium conditioned by these cells. Within each EB induced by medium conditioned by precardiac endoderm/mesoderm, over 80% of the cells become cardiac myocytes. The inductive efficacy of medium conditioned by avian precardiac endoderm/mesoderm provides a platform to biochemically define factors that induce cardiac myocyte differentiation in ES cells, and provides a platform for developing other methods for directing the development of particular cells from stem cells.

### CROSS REFERENCE TO RELATED APPLICATION

This Application claims priority from U.S. provisional patent application Ser. No. 60/581,946 filed Jun. 22, 2004.

### STATEMENT REGARDING FEDERALLY SPONSORED RESEARCH

This invention was made with United States government support from the National Institutes of Health (NIH) grant numbers NIH HL39829-16A1S1 (JL) and NIH T 32 HL07792 (DRR). The United States government has certain rights in this invention.

### FIELD OF THE INVENTION

The present invention relates to protocols to induce stem cells. Specifically, the invention relates to a method for induction of cardiac myocytes in embryonic stem (ES) cells, utilizing mechanistic cues from the embryonic processes that normally regulate the development of the heart in the embryo.

### BACKGROUND OF THE INVENTION

The potential of adult and embryonic stem (ES) cells to regenerate adult tissue has caused extraordinary interest in their therapeutic application. Adult stem cells, residing within niches of mature tissues and in bone marrow are, respectively, considered unipotent and multipotent with regard to their ability to form various cell types. Many instances of adult stem cells' ability to differentiate and become incorporated into adult tissues have been previously disclosed. By contrast ES cells, which are derived from the inner cell mass of blastocyst-stage embryos, are pluripotent, due to their potential to differentiate into all of the more that 200 cell types present in adults.

The therapeutic application of ES cells to regenerate damaged or diseased adult tissues is of high interest. However, such therapeutic utilization requires the resolution of formidable issues including the prevention of immune rejection, assurance that ES cells do not contain infectious agents, and the ability of ES cells to become highly differentiated into the cell type one wishes to repair. Hence the ability to differentiate ES cells to desired cell types is paramount.

The ability to regenerate damaged myocardium with new cardiac myocytes is a major objective of cardiac care, not only to minimize damage following acute pathological cardiac events but also to treat chronic cardiac insufficiency. Hence, generating cardiac myocytes from ES cells has therapeutic appeal. Normally, human and mouse ES cells spontaneously differentiate into cardiac myocytes; unfortunately however, the percentage of spontaneously differentiated cardiac myocytes in populations of ES cells is unacceptably low (˜10%), not only due to their presence in insufficient numbers to effect repair but also due to the high incidence (˜90%) of non-cardiac cells, some of which may form tumors. Therapeutic utilization of ES cells demands that they must be differentiated into cells of the desired target tissue at levels approaching 100%, in order to maximize regenerative efficacy while minimizing potential for tumor formation. Despite the high level of scientific activity in this area of endeavor, to date, no one has achieved direct induction of cardiac myocytes from ES cells via factors that induce heart development in the embryo.

U.S. Pat. No. 6,818,210 describes methods for cellular grafting in myocardial tissue of an animal, comprising forming a stable graft of embryonic cardiomyocyte cells. It also describes a method to manufacture essentially pure populations of cardiac myocytes from ES cells that may be used for such grafts. This method utilizes a positive selection in which ES cells are engineered such that upon their spontaneous differentiation into cardiac myocytes, they manufacture a neomycin-resistance protein that confers resistance to an antibiotic which kills non-myocyte cells in the same culture.

U.S. patent application No. 20050037489 describes a method of generating cells displaying at least one characteristic associated with a cardiac phenotype. The method involves (a) partially dispersing a confluent cultured population of human stem cells, thereby generating a cell population including cell aggregates; (b) subjecting the cell aggregates to culturing conditions suitable for generating embryoid bodies; (c) subjecting the embryoid bodies to culturing conditions suitable for inducing cardiac lineage differentiation in at least a portion of the cells of said embryoid bodies, said culturing conditions suitable for inducing cardiac lineage differentiation including adherence of said embryoid bodies to a surface, and culture, medium supplemented with serum, thereby generating cells predominantly displaying at least one characteristic associated with a cardiac phenotype. A drawback of the described method is that it relies on direct selection of spontaneously-appearing ES-derived cardiac myocytes, does not provide a high level of homogeneity of the cardiac myocyte population, and defines cardiac myocytes as cells exhibiting “at least one characteristic associated with a cardiac phenotype.”

U.S. patent application No. 20040106095 discloses cultured human embryonic stem cells that form embryoid bodies, some of which contain spontaneously-appearing cardiac myocytes that, based on electrical characteristics, represent the major types of cardiac myocytes (ventricular, atrial, nodal) present in the heart. However, methods of inducing homogeneous populations of highly differentiated cardiac myocytes are not addressed. The application describes phenomena occurring only in those myocytes which spontaneously appear under standard culture conditions in some embryoid bodies.

U.S. patent application No. 20040033214 describes methods of purifying pluripotent embryonic-like stem cells and compositions, cultures and clones thereof. The publication also describes a method of transplanting the pluripotent stem cells into a mammalian host, such as human, by introducing the stem cells into the host. The publication also describes to methods of producing mesodermal, endodermal or ectodermal lineage-committed cells by culturing or transplantation of the pluripotent embryonic-like stem cells. Application No. 20040033214 addresses “embryonic-like” stem cells, which are actually adult stem cells. Although the issue of whether such “embryonic-like” adult stem cells may be differentiated into cardiac myocytes is peripherally addressed, issues concerning these cells' homogeneity, and the efficiency with which they become fully differentiated into beating myocytes, are not addressed. Moreover, application No. 20040033214 does not utilize cues based on embryological factors for inducing cellular differentiation.

A recent report by Xu et al. describes use of a density gradient to isolate spontaneously-appearing cardiac myocytes from embryoid bodies cultivated under standard conditions. By this means of direct selection, a 70%-enriched population of ES-derived cardiac myocytes was achieved. The major drawback to this approach is that, in addition to the laborious task of manipulating cells on a density gradient, a large number of embryoid bodies is required due to the small percentage of spontaneously-arising myocytes within each embryoid body.

### SUMMARY OF THE INVENTION

The invention provides a method to improve the incidence of cardiac myocyte differentiation in a fashion that overcomes drawbacks of existing methods.

A drawback of U.S. Pat. No. 6,818,210 is the requirement for positive selection for pre-engineered ES cells. Advantageously, the present invention provides a method to directly induce a highly purified population of ES-derived cardiac myocytes which does not involve positive selection or the use of antibiotics. Rather, the present invention provides a method by which ES-derived cardiac myocytes are obtained via direct induction with normally occurring factors secreted by the same embryonic tissues that induce heart development in the embryo.

Alternative to using physical separation methods to isolate spontaneously differentiating myocytes, the invention utilizes direct induction to obtain a homogeneous population of ES-derived cardiac myocytes. Benefits of direct induction used according to the method of the invention include acquiring significantly larger numbers of myocytes than can be obtained by direct selection, using a method that is significantly less laborious. The technique of direct induction disclosed herein is based on the mechanism of cardiac myocyte induction which normally occurs during development of the heart within the embryo. The inventors have demonstrated that gastrulation-stage anterior lateral endoderm from chick embryos, hereafter termed ‘precardiac endoderm’ (preE), potently induces cardiac myocyte differentiation in embryonic precardiac mesoderm; subsequently the extraordinary cardiogenic potency of preE was shown by its ability to induce differentiation of cardiac myocytes within non-precardiac mesoderm. The examples herein demonstrate that, when used as a feeder layer, chick preE and/or its associated tissues induce cardiac myocyte differentiation in co-cultured mouse embryonic stem (mES) cells. Specifically, when co-cultured with preE, approximately 65% of embryoid bodies exhibit expression of cardiac markers followed by rhythmic contractility. Most remarkably, it is furthermore disclosed that co-culture of ES cells with preE plus precardiac mesoderm (preM), or replacement of this combination of explants with cell-free medium conditioned by it, respectively induces rhythmic contractility in 100% or 92% of EBs; importantly, the latter are highly enriched (˜86%) in cardiac myocytes.

The invention provides a method to utilize cues, as informed from mechanisms of development which normally occur in the embryo, to direct induction. Toward this end, cues from mechanisms that regulate the normal embryonic development of tissues that would be desirably regenerated in the adult should assist in the design of approaches to induce ES cells into specific cell types.

Another advantage of the present invention is that the resulting cardiac myocytes express a cardiac myocyte marker (i.e. cardiac myosin heavy chain) and also demonstrate rhythmic, synchronous contractility, such physiologic activity indicating the expression and subsequent organization of tens to hundreds of proteins which must cooperate to establish a functional cardiac myocyte.

The present invention also uses normally occurring inductive factors to achieve unanimously contractile embryoid bodies containing a highly purified (>80%) population of cardiac myocytes.

The invention provides a method that can be used with efficacy both in vitro and in vivo.

The invention provides a method to demonstrate that cell-cell contact is not necessary for the cardiogenic effect induced by secretions of precardiac endoderm+mesoderm.

Furthermore, the disclosed methods provide a framework or platform for extending this approach to include the induction of ES cells into the cellular phenotypes of organs other than the heart, using rationales based on the cues provided by the respective embryonic mechanisms which regulate the development of said cell types in the embryo.

The invention provides a method for the induction of cardiac myocytes from embryonic stem cells comprising the following steps: (i) expanding cultures of ES cells to approximately 60% confluency in a defined medium; (ii) dispersing said stem cells in suspension culture conditions to provide suspended cell aggregates; (iii) growing cell aggregates to form pre-embryoid spheres (preEBs); (iv) selecting said preEBs of a size within an optimal size range of about 170-230 μm; and (v) subjecting said selected preEBs to culturing conditions suitable for induction of cardiac myocytes.

### BRIEF DESCRIPTION OF THE DRAWING FIGURES

FIG. 1 shows a schematic representation of the protocol for cardiomyogenesis, on cue, established by the co-inventors to (*) inhibit spontaneous differentiation of mES cells to other cell types while (**) inducing cardiac myocyte differentiation. This figure depicts the three phases of an embodiment of a method of the invention: expansion, suspension, and induction. During expansion and suspension spontaneous differentiation of the stem cells is strategically prevented. Induction culminates in terminal cardiac differentiation.

FIG. 2 shows photographic images from culture dishes, demonstrating that the size of pre-embryoid bodies (pre-EBs) selected for the cardiac myocyte induction phase is important. Pre-EBs in suspension phase were selected based on size and induced with either preE+M-cm (i.e. medium conditioned by precardiac endoderm and precardiac mesoderm; panels a & b) or fresh non-conditioned medium which was used as a control (panel c). The pre-EB in panel (a) was induced after two days in suspension phase after having grown to a diameter of ˜200 μm, and shows the coherent morphology of this EB, which contained many homogeneous groups of rhythmically contractile cells. Panel (b) shows an EB from a ˜400 μm diameter pre-EB; by contrast this EB had very few contractile cells, and displayed protuberances containing non-cardiac cells. Panel (c) shows an EB generated from a ˜200 μm diameter pre-EB plated in fresh, non-conditioned medium; this EB exhibited pronounced outgrowths (arrows) with little evidence of contractile activity. All panels are shown at the same magnification; the bar in (c)=100 μm.

FIG. 3 shows photographic images of immunohistochemically stained cells in culture dishes, indicating that control conditions favor differentiation toward a neuronal phenotype. Pre-EBs of 200 μm diameter were plated alone on a fibronectin substrate (No Cell Control, panels a-d), or with non-precardiac posterior endoderm (PostE Control, panels e-h). After eight days, EBs in panels (b) and (f) were immunostained to detect β-Tubulin Type III, a molecular marker for neurons, revealing extensive differentiation of neuronal cells. By contrast, staining for sarcomeric myosin heavy chain (s-MHC, panels d,h) detected very few, and small, areas of spontaneously appearing cardiac myocytes. (see arrow in panel h). Staining with propidium iodide (PI) was used to mark the nuclei in all cells, whether neurons or myocytes, in the cultured (panels a,c,e,g). All panels are at the same magnification; bar in h=100 μm.

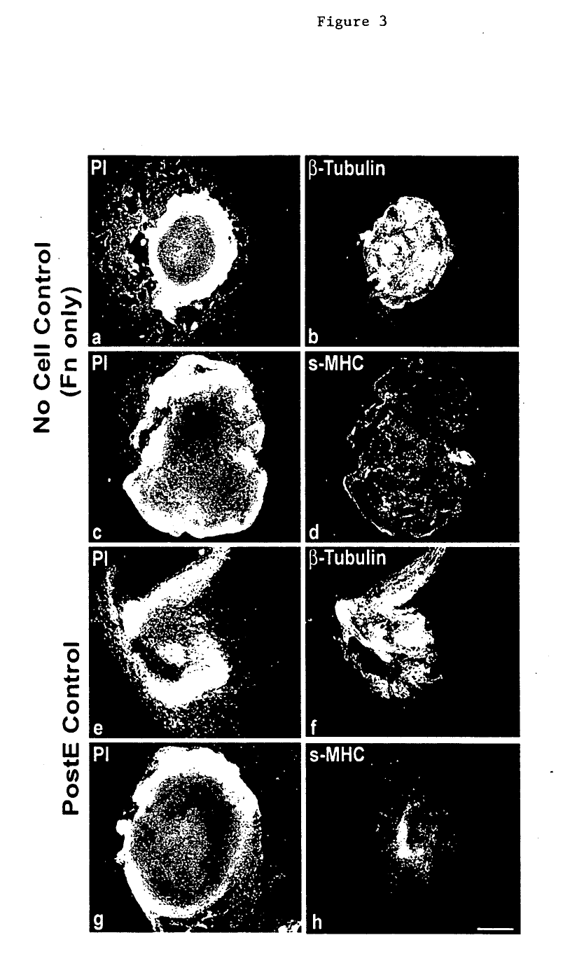

FIG. 4 presents three photographic images from immunohistochemically stained cells in culture dishes showing that precardiac endoderm (preE) induces cardiac gene expression in differentiating EBs. Pre-EBs were co-cultured with avian preE for four days, then immunostained to detect sarcomeric MHC. Panel (a) shows portions of two EBs (each encircled by a broken line) in the same culture; intense immunostaining of MHC was detected throughout the EB on the right, which is shown in its entirety in panel (b) and at higher magnification in panel (c). Although beating was not observed at this stage (induction Day 4), the arrows in panel (c) denote sarcomeres, structures that medium cellular contraction. Bars=100 μm in (a) & (b), 10 μm in (c).

FIG. 5 shows photographic images of embryonic stem (ES) cells growing in culture and exhibiting lacZ staining, which is indicative of cardiac myocyte differentiation. The ES cells used in this experiment, termed αMHC-lacZ ES cells, were engineered such that the lacZ signal is manufactured by a cardiac-specific promoter (α-Myosin Heavy Chain), indicating the exclusive presence of cardiac myocytes as apposed to other muscle cell types (skeletal, smooth). Pre-EBs from αMHC-lacZ mES cells were co-cultured as indicated in the panels: (a), co-culture with precardiac endoderm (preE); (b), co-culture with posterior (i.e. non-precardiac) endoderm (postE); (c), co-culture with precardiac endoderm plus mesoderm (preE+M); (d), no cell control. After seven days, cultures were histochemically reacted to detect LacZ as indicative of αMHC gene expression, hence cardiac myocyte identity. All panels are at the same magnification; bars in (a) & (c)=100 μm.

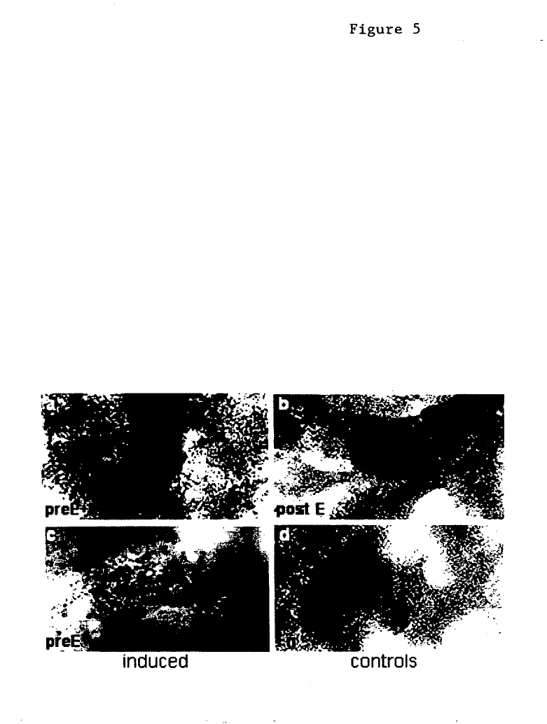

FIG. 6 is a graphical representation of the incidence of EBs that exhibit beating cardiac myocytes as a function of candidate inducers present in the culture environment. Panel a: Effect of embryonic cells per se on EB differentiation. Panel b: Effect of medium conditioned by embryonic cells on EB differentiation. Pre-EBs were co-cultured up to 21 days with the indicated cells or embryonic explants (panel a), or with medium conditioned by explanted preE+M (panel b). As shown in (a), control conditions consisting of pre-EBs incubated on fibronectin only (i.e. a “no cell” control), or a feeder layer of mouse embryonic fibroblasts (fibronectin/MEF; a “cell” control), or a feeder layer of non-precardiac posterior endoderm (postE), did not cause appreciable differentiation in co-cultured pre-EBs. By contrast, ˜65% of pre-EBs co-cultured with chick precardiac endoderm (preE) became rhythmically contractile; moreover, inclusion of precardiac mesoderm (preM), which by itself has potency equal to pre E, along with preE resulted in 100% rhythmically contractile explants (not shown). As shown in panel b, cell-free medium conditioned by preE+M (see ‘explant-conditioned medium) induced cardiogenesis in nearly all (˜100%) EBs, whereas fresh (non-conditioned) avian explant medium had less than 10% efficacy. The result in panel b demonstrates that cell-cell contact is not necessary for the cardiogenic effect induced by secretions of precardiac endoderm+mesoderm.

FIG. 7 is a photographic series from confocal microscopic views of cells within EBs that were immunostained to detect cardiac myocytes. To evaluate the percentage of cardiac myocytes in induced and uninduced EBs, intact EBs were fixed on Induction Day 7 and stained for MHC, which detects cardiac myocytes, and propidium iodide (PI) which marks all cells, whether differentiated or not. As shown schematically in (a) and (b), cardiac myocytes in each EB were enumerated in ten non-overlapping confocal fields [five x-y fields shown as the rectangles in panel a, at two z-depths (dotted lines in panel b)] taken using the 100×objective. To demonstrate the distribution of myocytes throughout EBs grown under each experimental condition, three images from each EB are shown. Panels (c-e) depict three sections taken through a no cell (i.e. fresh medium) control EB, panels (f-h) depict three sections through a postE control EB, and panels (i-k) show three sections through an EB induced by preE+M-cm. Note the homogenous distribution of myocytes (green) in sections i-k, in contradistinction to the sporadic myocyte groups in panels c-h. Panel 1 shows the percentage of MHC-stained cells in five EBs grown under each condition, revealing respective averages of 7.1%, 16.3% and 85.6% myocytes in No Cell, PostE and preE+M-cm EBs (p<0.001; T-Test); to verify this result, triplicate EBs dis-aggregated after two weeks' induction with preE+M-cm and re-plated at non-confluent density revealed an average of 74% myosin-positve cells (m; bar=10 μm).

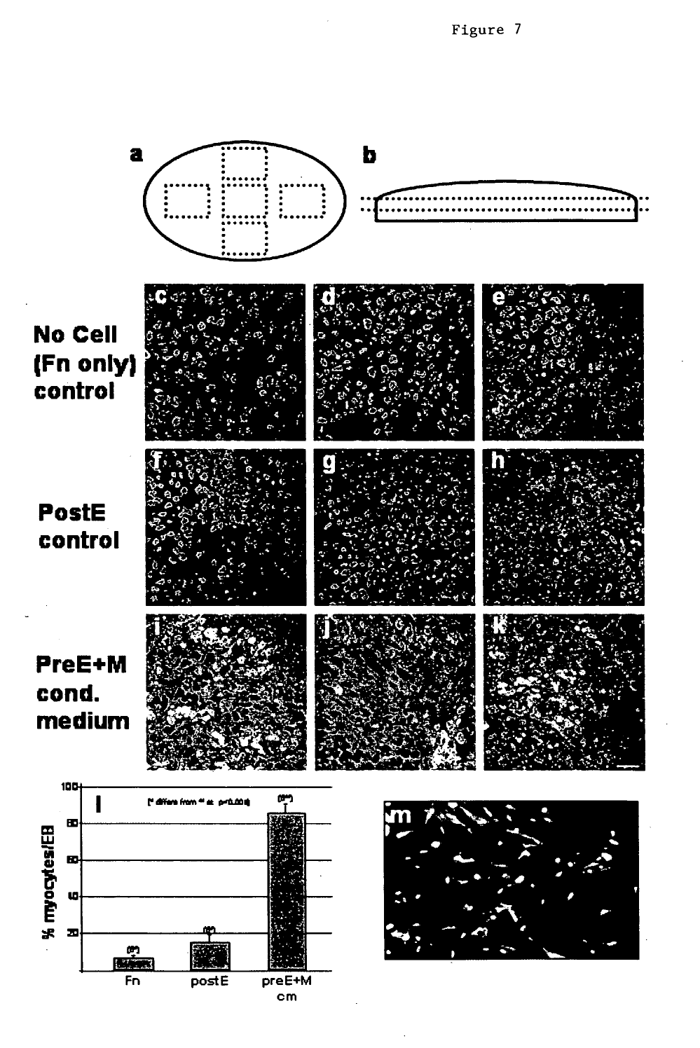

### DETAILED DESCRIPTION OF THE INVENTION

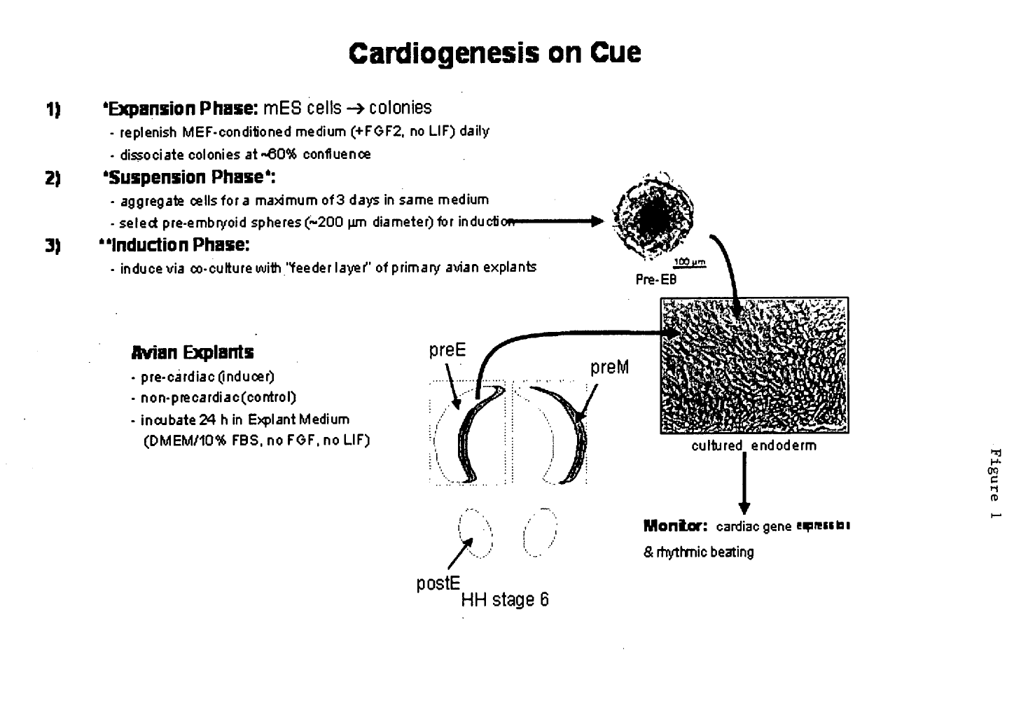

It is described here for the first time that embryonic stem (ES) cells can be induced to form cardiac myocytes with high levels of enrichment for rhythmically contractile myocytes and that the method of the invention that induces such cardiac myocyte differentiation also inhibits spontaneous differentiation of ES to other cell types. We call this invention “Cardiomyogenesis on Cue.” FIG. 1 shows a general embodiment of a method according to the invention. This figure depicts the three phases of the method: (1) expansion phase, (2) suspension phase, and (3) induction phase. During the expansion and suspension phases, spontaneous differentiation of the stem cells is strategically prevented. Induction culminates in terminal cardiac differentiation.

It is known that cardiac myocytes can be found in embryoid bodies formed from ES cells, having arisen by spontaneous differentiation,. In such embryoid bodies, stem cell differentiation into a variety of tissue types typically occurs. In the absence of molecular guidance provided by using cues akin to those that regulate natural development during embryology, the spontaneous incidence of cardiac myocytes occurs at a therapeutically disappointing low number (i.e. ˜10% of total cells in the embryoid body).

By using the same cells that induce the heart in an embryo, the present method achieves a very high degree of cardiac myocyte enrichment in ES cells. There are a number of known techniques that give rise to the formation of embryoid bodies, and, the methods used for forming the EBs result in a greater or lesser percentage of EBs containing some cardiac myocytes. According to the invention, exploitation of normal developmental cues vastly improves the incidence of cardiac myocytes, in a predictable manner of time and yield. Because this technique indicates an approach to obtain homogeneous populations of cardiac myocytes from ES cells, clinical application of the latter may be facilitated.

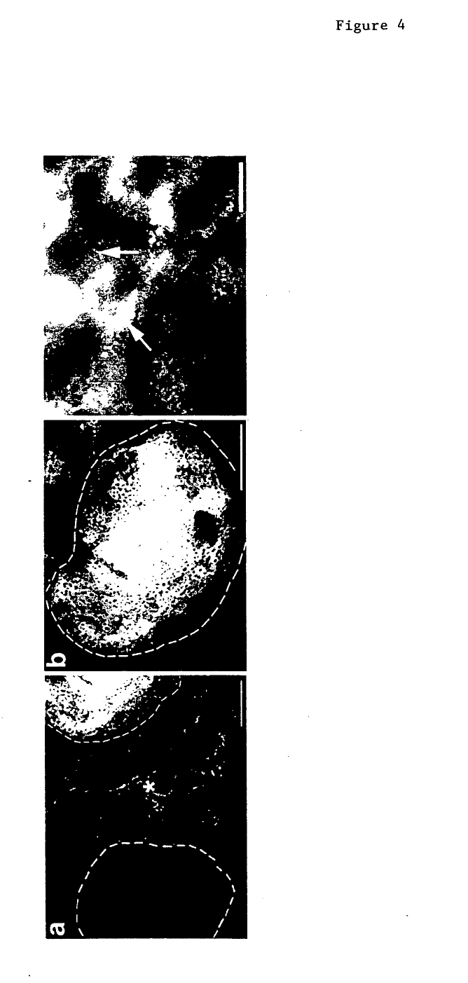

The potency of embryonic precardiac endoderm (preE), but not posterior endoderm (postE), to induce terminal cardiac myocyte differentiation in co-migrating precardiac mesoderm cells in the embryo is known. According to the invention, the inclusion of precardiac mesoderm with endoderm (preE+M) strongly increases the incidence of cardiogenesis in ES cells, causing substantially all EBs to become rhythmically contractile. The effect of co-culturing pre-EBs with precardiac endoderm (preE), which specifies and induces differentiation of precardiac mesoderm in the embryo, is shown in FIG. 4. Moreover, cell-free medium conditioned by preE+M (preE+M-cm) retains the cardiogenic potency of the explants. The latter findings demonstrate that contact between inducing and responding cells is not required for cardiac differentiation, and that all cardiac myocytes are derived from ES cells since there are no embryonic explants in the cultures containing conditioned medium.

An embodiment of the method comprises three steps—expansion, suspension, and induction—the latter culminating in terminal cardiac myocyte differentiation. During expansion and suspension phases, the method prevents spontaneous differentiation of mES cells, so that cardiac differentiation can be induced on cue during the induction phase.

To prevent spontaneous differentiation during the expansion phase, ES cells are expanded on a layer of MEF cells in MEF-conditioned medium which contains no LIF (leukemic inhibitory factor) but which does contain FGF-2 (fibroblast growth factor-2). Importantly, LIF, which is conventionally used to prevent spontaneous differentiation of ES cells, is withheld because this factor inhibits cardiac myocyte differentiation. And, FGF-2 was included because this factor was deemed to (i) inhibit differentiation of pluripotent cells and (ii) promote cardiac myocyte differentiation once this process is induced by other unknown factors. According to the invention ES cells are grown to no greater than 60% confluence during the expansion phase, since increasing confluency (i.e. the extent of cellular density in the culture) promotes spontaneous differentiation.

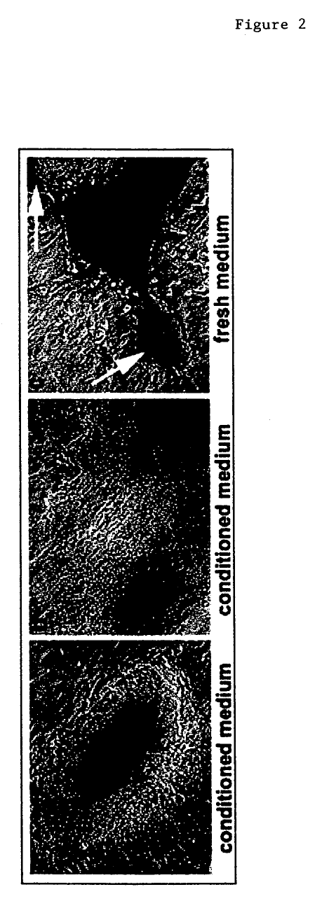

To initiate the suspension phase, the same growth conditions are used except that the MEF feeder layer and the fibronectin coating on the culture dish surface are removed. As a result, ES cells continue to grow during their literal suspension in the growth medium. Because the ES cells cannot adhere to the culture dish surface, they adhere to each other, resulting in the formation of multicellular spheres termed “pre-embryoid bodies”. To achieve optimal cardiac myocyte differentiation during the next (induction) phase, the suspension phase must last for only a maximum of 2-3 days (in contradistinction to 7+days as employed in standard protocols), and, that pre-EBs selected for the induction phase must not exceed about 200 μm (±15%) diameter (see FIG. 2).

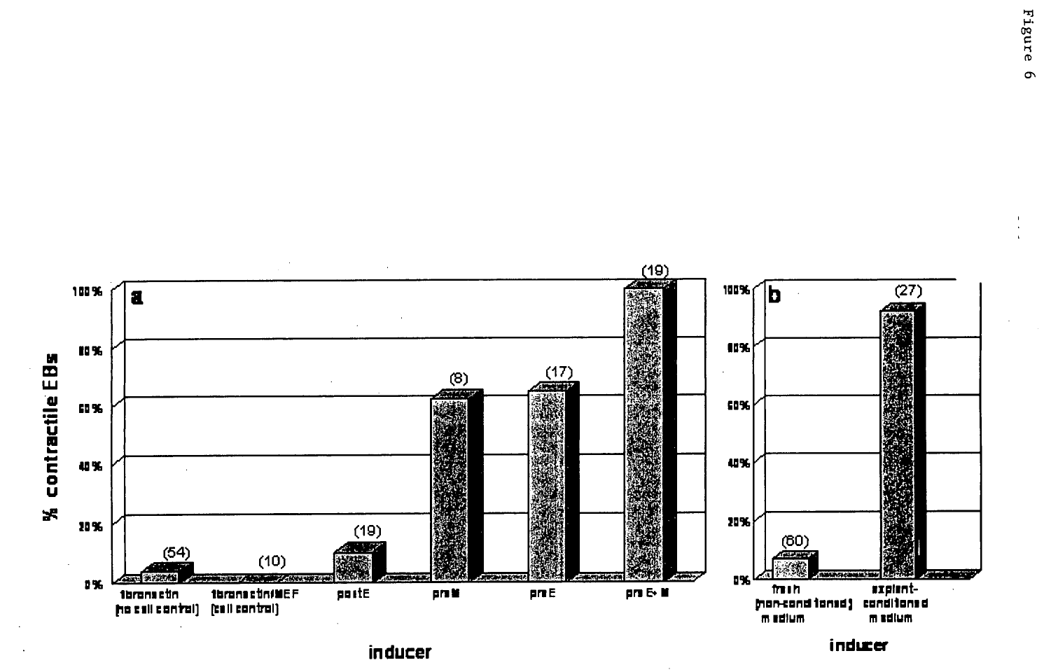

To initiate the induction phase during which cardiac myocyte differentiation is induced, suspended pre-EBs are individually implanted adjacent to, or directly upon, explanted avian precardiac endoderm (preE) and/or mesoderm (preM) cells (see tabulated results, FIG. 6a), or, cultured in medium previously conditioned by explanted precardiac endoderm+mesoderm cells (preE+preM-cm; see tabulated results, FIG. 6b). The selection of pre-EBs from the suspension phase for this purpose is restricted, such that the pre-EBs are approximately 200 μm (±30 μm) in diameter, thereby containing approximately 1,000 cells. Adherence to this criterion results in optimal cardiac morphogenesis approximately seven days after the onset of induction, as defined by the development of coherent embryoid bodies that contain multiple foci of rhythmically contractile cardiac myocytes.

Cell to cell contact is not required to induce cardiac myocyte differentiation, as demonstrated by the ability of cell-free medium conditioned by explanted endoderm and mesoderm to mimic the inductive effect of explanted cells (FIG. 6). However the chronology of differentiation induced by preE+M-cm is delayed when compared with induction by whole cells; for example, biochemical differentiation and onset of beating in pre-EBs are respectively observed, on average, six and nine days after induction with preE+M-cm, in comparison with four and seven days after induction with explants. Finally, although the increased potency of preE+M, in comparison with preE alone, to induce cardiogenesis in mES cells is unexplained, this result is consistent with findings indicating that stem cell differentiation toward specific cell-type endpoints is influenced by local environments.

In addition to its ability to induce heart development in the embryo, precardiac endoderm (preE) can induce cardiogenesis at ectopic sites in the embryo, indicating that preE has a role in the ‘specification’ of cells to the cardiac lineage, which in the embryo occurs during early gastrulation. It is believed that at the onset of gastrulation, as ingressing cells from the epiblast become diverged into endodermal and mesodermal germ layers, the endoderm specifies precardiac mesoderm within a subset of the mesoderm germ layer. It is further believed that these embryonic processes are mimicked during the conversion of pre-EBs to beating EBs when, under the influence of signals from preE+M that are sufficiently potent to over-ride competing signals that may be present to induce the differentiation of other cell types, cardiac myocytes are induced in highly enriched fashion.

The observation of rhythmic contractility in virtually all (100%; see FIG. 6) EBs induced by preE+M or by its conditioned medium (preE+M-cm) is unprecedented. Using an approach based on induction by an endoderm cell line (END-2 cells) that did not include cells of mesodermal origin or bona fide embryo cells, it has been reported that beating myocytes appeared in only in 35% of co-cultures, and, that each beating locus contained only 10-200 cardiac myocytes. And, although an enrichment of cardiac myocytes from ES cells of ˜70% has been obtained, this was achieved by physical selection rather than by embryonic (direct) induction. (Xu C. et al.: “Characterization and enrichment of cardiomyocytes derived from human embryonic stem cells.” Circ Res., vol. 91, 2002, pp 501-508.) Hence, the present method uniquely exploits constitutive signals of embryology to achieve a high degree of cardiac myocyte enrichment (over 80%) in ES cells, as obtained by inducing cardiac myocytes with the same embryonic cells that induce the heart in the embryo.

The present method to induce mouse ES cells to a cardiac phenotype utilizes culture conditions that can be seamlessly applied to human ES cells. Most significantly, LIF (leukemic inhibitory factor) is omitted from, and FGF (fibroblast growth factor) is added to, mouse embryonic fibroblast (MEF)-conditioned medium during the expansion and suspension phases to prevent spontaneous ES cell differentiation.

It is known that treatment of human ES cells with combinations of purified growth factors induces phenotypes having characteristics of specific embryonic germ layers; however, EBs enriched for single cell types are not generated. On the other hand, treatment of less primitive embryonic cells, from gastrulation and post-gastrulation stage embryos, with specific growth factor combinations is known to induce specific cell types including pituitary, neural crest, and cardiac myocytes (the latter having been discovered by the inventors). Rationally defined cocktails of purified growth factors, based on composition of the environment in which embryonic cells differentiate, may induce enriched cohorts of specific cell types from ES cells. The cardiogenic potency of preE+M, combined with the demonstration that preE+M can be replaced by its conditioned medium, provides a starting point for the biochemical identification of these cells' secretory products, with the goal of defining a cocktail of purified growth factors that can induce cardiogenesis in ES cells on cue.

### REFERENCES

1. Passier R, Mummery C. Origin and use of embryonic and adult stem cells in differentiation and tissue repair. Cardiovasc Res. 2003;58:324-335.
2. Odorico J S, Kaufman D S, Thomson J A. Multilineage differentiation from human embryonic stem cell lines. Stem Cells 2001;19:193-204.
3. Quaini F, Urbanek K, Beltrami A P, Finato N, Beltrami C A, Nadal-Ginard B, Kajstura J, Leri A, Anversa P. Chimerism of the Transplanted Heart. New England J Med. 2002;346:5-15.
4. Beltrami A P, Barlucchi L, Torella D, Baker M, Limana F, Chimenti S, Kasahara H, Rota M, Musso E, Urbanek K, Leri A, Kajstura J, Nadal-Ginard B, Anversa P. Adult cardiac stem cells are multipotent and support myocardial regeneration. Cell 2003;114:763-776.
5. Xu C, Police S, Rao N, Carpenter M K. Characterization and enrichment of cardiomyocytes derived from human embryonic stem cells. Circ Res. 2002;91:501-508. Xu C, Inokuma M S, Denham J, Golds K, Kundu P, Gold J D, Carpenter M K.
6. Kehat I, Gepstein A, Spira A, Itskovitz-Eldor J, Gepstein L. High-resolution electrophysiological assessment of human embryonic stem cell-derived cardiomyocytes: a novel in vitro model for the study of conduction. Circ Res. 2002;91:659-661.
7. Doevendans P A, Kubalak S W, An R H, Becker D K, Chien K R, Kass R S. Differentiation of cardiomyocytes in floating embryoid bodies is comparable to fetal cardiomyocytes. J Mol Cell Cardiol. 2000;32:839-851.
8. Boheler K R, Czyz J, Tweedie D, Yang H T, Anisimov S V, Wobus A M. Differentiation of pluripotent embryonic stem cells into cardiomyocytes. Circ Res. 2002;91:189-201.
9. Xu C, Inokuma M S, Denham J, Golds K, Kundu P, Gold J D, Carpenter M K. Feeder-free growth of undifferentiated human embryonic stem cells. Nat Biotechnol. 2001;19:971-974.
10. Lough J, Sugi Y. Endoderm and heart development. Dev. Dyn. 2002;217:327-342.
11. Schultheiss T M, Xydas S, Lassar A B. Induction of avian cardiac myogenesis by anterior endoderm. Development. 1995:121:4203-4214.
12. Sugi Y, Lough J. Anterior endoderm is a specific effector of terminal cardiac myocyte differentiation of cells from the embryonic heart forming region. Dev Dyn. 1994;200:155-162.
13. Wood S A, Allen N D, Rossant J, Auerbach A, Nagy A. Non-injection methods for the production of embryonic stem cell-embryo chimeras. Nature 1993;365:87-89.
14. Misra R P, Bronson S K, Xiao Q, Garrison W, Li J, Zhao R, Duncan S A. Generation of single-copy transgenic mouse embryos directly from ES cells by tetraploid embryo complementation. BMC Biotechnol. 2001;1:12.
15. Bader A, Gruss A, Hollrigl A, Al-Dubai H, Capetanaki Y, Weitzer G. Paracrine promotion of cardiomyogenesis in embryoid bodies by LIF modulated endoderm. Diff. 2001;68:31-43.
16. Hamburger V, Hamilton H L. A series of normal stages in the development of the chick embryo. J Morphol. 1951;88:49-92.
17. Rudy D E, Yatskievych T A, Antin P B, Gregorio C C. Assembly of thick, thin and titin filaments in chick precardiac explants. Dev Dyn. 2001;221:61-71.
18. Hogan B, Beddington R, Constantini F, Lacy E. Manipulating the mouse embryo: a laboratory manual, second edn. New York: Cold Spring Harbour Laboratory Press, 1994
19. Goldstein R S, Drukker M, Reubinoff B E, Benvenisty N. Integration and differentiation of human embryonic stem cells transplanted to the chick embryo. Dev Dyn. 2002;225:80-86.
20. Lough J, Barron M, Brogley M, Sugi Y, Bolender D L, Zhu X. Combined BMP-2 and FGF-4, but neither factor alone, induces cardiogenesis in non-precardiac embryonic mesoderm. Dev Biol. 1996;178:198-202.
21. Antin P B, Taylor R G, Yatskievych T A. Precardiac mesoderm is specified during gastrulation in quail. Dev Dyn. 1994;200:144-153.
22. Sugi Y, Lough J. Developmental expression of fibroblast growth factor receptor-1 (cek-1; flg) during heart development. Dev Dyn. 1995;202:115-125.
23. Maltsev V A, Rohwedel J, Hescheler J, Wobus A M. Embryonic stem cells differentiate in vitro into cardiomyocytes representing sinusnodal, atrial and ventricular cell types. Mech Dev. 1993;44:41-50.
24. Condorelli G, Borello U, De Angelis L, Latronico M, Sirabella D, Coletta M, Galli R, Balconi G, Follenzi A, Frati G, Cusella De Angelis M G, Gioglio L, Amuchastegui S, Adorini L, Naldini L, Vescovi A, Dejana E, Cossu G. Cardiomyocytes induce endothelial cells to trans-differentiate into cardiac muscle: implications for myocardium regeneration. PNAS 2001;98:10733-10738.
25. Eisenberg L M, Burns L, Eisenberg C A. Hematopoietic cells from bone marrow have the potential to differentiate into cardiomyocytes in vitro. Anat Rec. 2003;274A:870-882.
26. Garcia-Martinez V, Schoenwolf G C Primitive-streak origin of the cardiovascular system in avian embryos. Dev Biol. 1993;159:706-719.
27. Linask K K, Lash J W. Early heart development: dynamics of endocardial cell sorting suggests a common origin with cardiomyocytes. Dev Dyn. 1993;196:62-69.
28. Sugi Y, Sasse J, Lough J. Inhibition of precardiac mesoderm cell proliferation by antisense oligodeoxynucleotide complementary to fibroblast growth factor-2 (FGF-2). Dev Biol. 1993;157:28-37.
29. Mummery C, Ward-Van Oostwaard D, Doevendans P, Spijker R, Van Den Brink S, Hassink R, Van Der Heyden M, Opthof T, Pera M, De La Riviere A B, Passier R, Tertoolen L. Differentiation of human embryonic stem cells to cardiomyocytes: role of coculture with visceral endoderm-like cells. Circulation 2003;107:2733-2740.
30. Barron M, Gao M, Lough J. Requirement for BMP and FGF signaling during cardiogenic induction in non-precardiac mesoderm is specific, transient, and cooperative. Dev Dyn. 2000;218:383-393.
31. Alsan B H, Schultheiss T M. Regulation of avian cardiogenesis by Fgf8 signaling. Development. 2002;129:1935-1943.
32. Dell'Era P, Ronca R, Coco L, Nicoli S, Metra M, Presta M. Fibroblast growth factor receptor-1 is essential for in vitro cardiomyocyte development. Circ Res. 2003;93:414-420.
33. Schuldiner M, Yanuka O, Itskovitz-Eldor J, Melton D A, Benvenisty N. Effects of eight growth factors on the differentiation of cells derived from human embryonic stem cells. Proc Natl Acad Sci U S A. 2000;97:11307-11312.
34. Dasen J S, Rosenfeld M G. Combinatorial codes in signaling and synergy: lessons from pituitary development. Curr Opin Genet Dev. 1999;9:566-574.
35. Sieber-Blum M. Growth factor synergism and antagonism in early neural crest development. Biochem Cell Biol. 1998;76:1039-1050.
36. Behfar A, Zingman L V, Hodgson D M, Rauzier J M, Kane G C, Terzic A, Puceat M. Stem cell differentiation requires a paracrine pathway in the heart. FASEB J. 2002; 16:1558-1566.

1. A method for treating heart disease, comprising at least one of:
(i) regenerating damaged myocardium by cell transplant;
(ii) replacing damaged cardiac cells with new cardiac myocytes;
(iii) administering said new cardiac myocytes by one of direct injection, transplantation, or infusion into the patient's vascular system;
(iv) obtaining cardiac myocytes from embryonic stem (ES) cells by induction;
(v) directing development of stem cells to cardiac myocytes using defined culture conditions

2. The method in claim 1 wherein said embryonic stem cells used to obtain cardiac myocytes are engineered to express a transgene encoding a secretable myogenic protein upon direct cardiomyogenic induction;

3. The method in claim 1 wherein said embryonic stem cells used to obtain cardiac myocytes are engineered to express a transgene encoding a secretable myogenic protein upon direct cardiomyogenic induction and the myogenic factor is Fibroblast Growth Factor-8, Transforming Growth Factor-Beta-2, Bone Morphogenetic Protein-2, Nitric Oxide Synthase, or Insulin-Like Growth Factor-1;

4. The method in claim 1 wherein said embryonic stem cells used to obtain cardiac myocytes by induction are embryonic stem cell-lines.

5. The method in claim 1 wherein said defined culture conditions comprises co-cultivation with embryonic endoderm (or embryonic endodermal cells, or embryonic endodermal cell-lines).

6. The method in claim 1 wherein said defined culture conditions comprises co-cultivation with embryonic mesoderm (or embryonic precardiac mesoderm cells, or embryonic mesodermal cell-lines).

7. The method in claim 1 wherein said defined culture conditions comprises co-cultivation with a mixture of embryonic endoderm (or embryonic endodermal cells or embryonic endoderm cell-lines) and embryonic precardiac mesoderm (or embryonic precardiac mesodermal cells or embryonic mesodermal cell-lines).

8. The method in claim 1 wherein said defined culture conditions comprises co-cultivation with cell-free medium obtained from cultures of embryonic endoderm (or embryonic endodermal cells or embryonic endoderm cell-lines).

9. The method in claim 1 wherein said defined culture conditions comprises co-cultivation with cell-free medium obtained from cultures of embryonic mesoderm (or embryonic precardiac mesoderm cells or embryonic mesodermal cell-lines).

10. The method in claim 1 wherein said defined culture conditions comprises co-cultivation with the cell-free medium obtained from a mixed culture of embryonic endoderm (or embryonic endodermal cells or embryonic endoderm cell-lines) plus embryonic precardiac mesoderm (or embryonic precardiac mesoderm cells or embryonic mesodermal cell-lines).

11. A method for the induction of cardiac myocytes from embryonic stem (ES) cells comprising the following steps: (i) expanding cultures of ES cells to approximately 60% confluence in a medium; (ii) dispersing said stem cells in suspension culture conditions to provide suspended cell aggregates; (iii) growing the cell aggregates to form pre-embryoid spheres (preEBs); (iv) selecting said preEBs of a size within a range of about 170-230 μm; (v) subjecting said selected preEBs to culturing conditions suitable for induction of cardiac myocytes.

12. The method in claim 11 wherein step (v) comprises co-cultivating said selected preEBs with embryonic endoderm, embryonic endodermal cells, embryonic endodermal cell-lines, or a combination thereof.

13. The method in claim 11 wherein step (v) comprises co-cultivating said selected preEBs with embryonic mesoderm, embryonic precardiac mesoderm cells, embryonic mesodermal cell-lines, or a combination thereof.

14. The method in claim 11 wherein step (v) comprises co-cultivating said selected preEBs with a mixture of a) embryonic endoderm, embryonic endodermal cells, embryonic endoderm cell-lines, or a combination thereof, and b) embryonic precardiac mesoderm, embryonic precardiac mesodermal cells, embryonic mesodermal cell-lines, or a combination thereof.

15. The method in claim 11 wherein step (v) comprises co-cultivating said selected preEBs with a cell-free medium obtained from cultures of embryonic endoderm, embryonic endodermal cells, embryonic endoderm cell-lines, or a combination thereof.

16. The method in claim 11 wherein step (v) comprises co-cultivating said selected preEBs with a cell-free medium obtained from cultures of embryonic mesoderm, embryonic precardiac mesoderm cells, embryonic mesodermal cell-lines, or a combination thereof.

17. The method in claim 11 wherein step (v) comprises co-cultivating said selected preEBs with a cell-free medium obtained from a mixed culture of embryonic endoderm, embryonic endodermal cells, embryonic endoderm cell-lines, or a combination thereof, with embryonic precardiac mesoderm, embryonic precardiac mesoderm cells, embryonic mesodermal cell-lines, or a combination thereof.

18. The method in claim 11 wherein step (v) comprises co-cultivating said selected preEBs with at least one biochemical factor purified from cell-free media prepared according to claims 15, 16, 17.

19. A method for directing the induction of cardiac myocytes from murine embryonic stem (ES) cells, the method comprising: (a) cultivating stem cells in MEF-conditioned medium comprising FGF but not LIF) to approximately 60% confluence; (b) aggregating cells in suspension for up to about three days in said medium to produce pre-embryoid spheres (preEBs); (c) selecting preEBs of approximately 170-230 μm diameter; (d) subjecting said selected preEBs to culture conditions effective to induce cardiac myocytes, thereby generating an enrichment of cardiac myocytes in the cell population while inhibiting spontaneous differentiation of embryonic stem cells into cardiac myocytes.

20. A method for directing the induction of cells other than cardiac myocytes from embryonic stem (ES) cells, the method comprising: (a) cultivating stem cells in MEF-conditioned medium comprising FGF but not LIF) to approximately 60% confluence; (b) aggregating cells in suspension for up to about three days in said medium to produce pre-embryoid spheres (preEBs); (c) selecting preEBs of approximately 170-230 μm diameter; (d) subjecting said selected preEBs to culture conditions, based on normal embryonic signaling, effective to induce a particular cell phenotype, thereby generating an enrichment of the desired cell phenotype in the cell population while inhibiting spontaneous differentiation of embryonic stem cells to other phenotypes.
